(19) United States
(12) Patent Application Publication
(10) Pub. No.: US 2026/0137285 A1
(43) Pub. Date: May 21, 2026

(54) WEARABLE DISPLAY DEVICE AND ELECTRONIC DEVICE USING THE SAME

(71) Applicant: Samsung Display Co., LTD., Yongin-si (KR)

(72) Inventor: Won Sang PARK, Yongin-si (KR)

(73) Assignee: Samsung Display Co., LTD., Yongin-si (KR)

(21) Appl. No.: 19/261,056

(22) Filed: Jul. 7, 2025

(30) Foreign Application Priority Data
Nov. 21, 2024 (KR) 10-2024-0167107

(51) Int. Cl. A61B 5/021; A61B 5/145; H10K 30/60; H10K 50/818; H10K 50/828; H10K 59/121; H10K 59/123; H10K 59/40; H10K 65/00

(52) U.S. Cl. CPC A61B 5/021; A61B 5/14542; H10K 30/60; H10K 50/818; H10K 50/828; H10K 59/121; H10K 59/123; H10K 59/40; H10K 65/00

(57) ABSTRACT

A wearable display device includes a housing formed in a ring or cylindrical shape, a display panel that detects a first light-sensing signal by emitting and receiving light in a first direction and detects a second light-sensing signal by emitting and receiving light in a second direction and is disposed inside the housing in a ring or cylindrical shape, and a main driver circuit that detects a pulse wave signal of a user and measure a biometric information of the user using the first light-sensing signal, and senses a movement of a touch by a part of the user's body using the second light-sensing signal. The first direction is an inward direction toward a center of the ring or cylindrical shape, and the second direction is an outward direction of the housing.

### CROSS-REFERENCE TO RELATED APPLICATION(S)

This application claims priority to and benefits of Korean Patent Application No. 10-2024-0167107 under 35 U.S.C. § 119, filed on Nov. 21, 2024, in the Korean Intellectual Property Office (KIPO), the entire contents of which are incorporated herein by reference.

### BACKGROUND

### 1. Technical Field

The disclosure relates to a wearable display device that can measure biometric information such as blood pressure and heart rate, and an electronic device using the same.

### 2. Description of the Related Art

As the information-oriented society evolves, various demands for display devices are ever increasing. Display devices are being employed by a variety of electronic devices such as smart phones, digital cameras, laptop computers, table PCs, navigation devices, and smart televisions. Portable display devices or electronic devices such as smartphones and tablet PCs may be equipped with a variety of features including image capturing, fingerprint recognition, face recognition, etc.

Recently, as the healthcare industry emerges as an industry with great prospects, methods for acquiring biometric information on health more conveniently are being developed. For example, there is an attempt to apply a traditional oscillometric blood pressure measurement device to a portable blood pressure measurement device. Such a portable blood pressure measurement device requires a separate light source, a sensor and a display, and is carried by a user in addition to a portable smart phone or tablet PC, which is inconvenient.

Recently, efforts have been made to combine portable display devices such as smartphones and tablet PCs with portable blood pressure measurement devices. Besides, there is a need for methods for measuring various biometric information such as heart rate, heart rate variability, respiration, cardiovascular disease and oxygen saturation in addition to blood pressure using portable display devices such as wearable display devices.

### SUMMARY

Aspects of the disclosure provide a wearable display device and an electronic device using the same that can be worn on a user's body in a ring or cylindrical shape, such as a ring, a bracelet, a watch and a band, and can measure biometric information such as blood pressure, heart rate, and oxygen saturation.

Aspects of the disclosure also provide a wearable display device that outputs lights on both the inner and outer surfaces to detect reflected light from a user's body on the both surfaces, to detect the user's biometric information using the reflected light on the inner surface and detect the direction of the user's motion using the reflected light on the outer surface, and an electronic device using the same.

It should be noted that objects of the disclosure are not limited to the above-mentioned object; and other objects of the disclosure will be apparent to those skilled in the art from the following descriptions.

According to an embodiment of the disclosure, a wearable display device may include a housing formed in a ring or cylindrical shape, a display panel that detects a first light-sensing signal by emitting and receiving light in a first direction and detects a second light-sensing signal by emitting and receiving light in a second direction and is disposed inside the housing in a ring or cylindrical shape, and a main driver circuit that detects a pulse wave signal of a user and measures a biometric information of the user using the first light-sensing signal, and senses a movement of a touch by a part of the user's body using the second light-sensing signal. The first direction may be an inward direction toward a center of the ring or cylindrical shape, and the second direction may be an outward direction of the housing.

In an embodiment, the wearable device may further include a scan driver that provides gate scan signals sequentially to first and second light-emitting pixels and first and second light-sensing pixels arranged in the display panel to control light-emitting timing of the first and second light-emitting pixels and light-receiving timing of the first and second light-sensing pixels, a circuit board electrically connected to the display panel, the main driver circuit mounted being mounted on the circuit board, a sensing circuit disposed on the housing or the circuit board to sense a movement direction of the user and a pressure applied by the user, and a memory that stores and sends the pulse wave signal of the user and the biometric information of the user.

In an embodiment, the display panel may include a biological signal detection area where at least one first light-emitting pixel that emits light in the first direction and at least one first light-sensing pixel that generates the first light-sensing signal based on an amount of light reflected from another part of the user's body in the first direction are located, and a touch detection area including at least one light-detecting area where at least one second light-emitting pixel and at least one second light-sensing pixel are disposed, and an image display area where a plurality of pixels for displaying images are disposed. The at least one second light-emitting pixel may emit light in the second direction, and the at least one second light-sensing pixel may generate the second light-sensing signal based on an amount of light reflected from the part of the user's body in the second direction.

In an embodiment, the at least one light-detecting area may be parallel to the image display area in a vertical or horizontal direction.

In an embodiment, the at least one first light-sensing pixel may include a photo-detecting part that outputs a light-sensing current proportional to an amount of received light incident in the first direction, and a sense driver that provides the first light-sensing signal proportional to an amount of the light-sensing current from the photo-detecting part to the main driver circuit. The photo-detecting part may include a photo-detector for generating and outputting the light-sensing current proportional to the amount of the received light, and the photo-detector may provide the light-sensing current proportional to the amount of the received light in the first direction to the sense driver.

In an embodiment, the at least one first light-emitting pixel may include a first light-emitting unit that emits light in a green wavelength range, and a pixel driver that applies a driving current to a first light-emitting element of the first light-emitting unit. The first light-emitting unit may be formed in a circular or polygonal shape in a plan view and emit light in the first direction.

In an embodiment, the first light-emitting unit may be formed in a circular or polygonal shape in a plan view and surround a photo-detector formed in a circular or polygonal shape in a plan view in the at least one first light-sensing pixel.

In an embodiment, the first light-emitting element of the at least one first light-emitting pixel may be formed by sequentially stacking a pixel electrode, an organic material layer and a common electrode to emit light in the first direction, the pixel electrode may be formed of at least one of a reflective metal material in which aluminum and titanium are sequentially stacked, a reflective metal material in which aluminum and indium tin oxide (ITO) are sequentially stacked, and a reflective metal material in which an alloy of silver, palladium and copper, and ITO are sequentially stacked, and the common electrode may be formed of at least one of a transparent metal material comprising ITO or IZO, a semi-transparent metal material comprising at least one of magnesium and silver, and a semi-transparent metal material comprising an alloy of magnesium and silver.

In an embodiment, the at least one second light-emitting pixel may include a second light-emitting unit that emits light, and a pixel driver that applies a driving current to a second light-emitting element of the second light-emitting unit. The second light-emitting unit may be formed in a circular or polygonal shape in a plan view and emit light in the second direction.

In an embodiment, the second light-emitting element of the at least one second light-emitting pixel may be formed by sequentially stacking a pixel electrode, an organic material layer and a common electrode to emit light in the second direction, the pixel electrode may be formed of at least one of a transparent metal material comprising ITO or IZO, a semi-transparent metal material comprising at least one of magnesium and silver, and a semi-transparent metal material comprising an alloy of magnesium and silver, and the common electrode may be formed of at least one of a reflective metal material in which aluminum and titanium are sequentially stacked, a reflective metal material in which aluminum and indium tin oxide (ITO) are sequentially stacked, and a reflective metal material in which an alloy of silver, palladium and copper, and ITO are sequentially stacked.

In an embodiment, the touch detection area may include first and second light-detecting areas adjacent to the image display area, the first light-detecting area may be located on a side of the image display area, and the second light-detecting area may be located on another side of the image display area, and the at least one second light-emitting pixel that emits light in the second direction and the at least one second light-sensing pixel that senses light incident in the second direction may be alternately arranged in the first and second light-detecting areas.

In an embodiment, each of the at least one second light-emitting pixel may include a second light-emitting unit that emits light, and a pixel driver that applies a driving current to a second light-emitting element of the second light-emitting unit. The second light-emitting unit may be formed in a circular or polygonal shape in a plan view and emit light in the second direction.

In an embodiment, the second light-emitting element of the at least one second light-emitting pixel may be formed by sequentially stacking a pixel electrode, an organic material layer and a common electrode to emit light in the second direction, the pixel electrode may be formed of at least one of a transparent metal material comprising ITO or IZO, a semi-transparent metal material comprising at least one of magnesium and silver, and a semi-transparent metal material comprising an alloy of magnesium and silver, and the common electrode may be formed of at least one of a reflective metal material in which aluminum and titanium are sequentially stacked, a reflective metal material in which aluminum and indium tin oxide (ITO) are sequentially stacked, and a reflective metal material in which an alloy of silver, palladium and copper, and ITO are sequentially stacked.

According to another embodiment of the disclosure, an electronic device may include a wearable display device. The wearable display device may include a housing formed in a ring or cylindrical shape, a display panel that detects a first light-sensing signal by emitting and receiving light in a first direction and detects a second light-sensing signal by emitting and receiving light in a second direction and is disposed inside the housing in a ring or cylindrical shape, and a main driving circuit that detect a pulse wave signal of a user using the first light-sensing signal, measures a biometric information of the user, and senses a movement of a touch by a part of the user's body using the second light-sensing signal. The first direction may be an inward direction toward a center of the ring or cylindrical shape, and the second direction may be an outward direction of the housing.

In an embodiment, the display panel may include a biological signal detection area where at least one first light-emitting pixel that emits light in the first direction and at least one first light-sensing pixel that generates the first light-sensing signal based on an amount of light reflected from another part of the user's body in the first direction are located, and a touch detection area including at least one light-detecting area where at least one second light-emitting pixel and at least one second light-sensing pixel are disposed, and an image display area where a plurality of pixels for displaying images is disposed. The at least one second light-emitting pixel may emit light in the second direction, and the at least one second light-sensing pixel may generate the second light-sensing signal based on an amount of light reflected from the part of the user's body in the second direction.

In an embodiment, the at least one first light-emitting pixel may include a first light-emitting unit that emits light in a green wavelength range, and a pixel driver that applies a driving current to a first light-emitting element of the first light-emitting unit. The first light-emitting unit may be formed in a circular or polygonal shape in a plan view and emits light in the first direction.

In an embodiment, the first light-emitting element of the at least one first light-emitting pixel may be formed by sequentially stacking a pixel electrode, an organic material layer and a common electrode to emit light in the first direction, the pixel electrode may be formed of at least one of a reflective metal material in which aluminum and titanium are sequentially stacked, a reflective metal material in which aluminum and indium tin oxide (ITO) are sequentially stacked, and a reflective metal material in which an alloy of silver, palladium and copper, and ITO are sequentially stacked, and the common electrode may be formed of at least one of a transparent metal material comprising ITO or IZO, a semi-transparent metal material comprising at least one of magnesium and silver, and a semi-transparent metal material comprising an alloy of magnesium and silver.

In an embodiment, the at least one second light-emitting pixel may include a second light-emitting unit that emits light, and a pixel driver that applies a driving current to a second light-emitting element of the second light-emitting unit. The second light-emitting unit may be formed in a circular or polygonal shape in a plan view and emits light in the second direction.

In an embodiment, the second light-emitting element of the at least one second light-emitting pixel may be formed by sequentially stacking a pixel electrode, an organic material layer and a common electrode to emit light in the second direction, the pixel electrode may be formed of at least one of a transparent metal material comprising ITO or IZO, a semi-transparent metal material comprising at least one of magnesium and silver, and a semi-transparent metal material comprising an alloy of magnesium and silver, and the common electrode may be formed of at least one of a reflective metal material in which aluminum and titanium are sequentially stacked, a reflective metal material in which aluminum and indium tin oxide (ITO) are sequentially stacked, and a reflective metal material in which an alloy of silver, palladium and copper, and ITO are sequentially stacked.

In an embodiment, the touch detection area may include first and second light-detecting areas adjacent to the image display area, the first light-detecting area may be located on a side of the image display area, and the second light-detecting area is located on another side of the image display area, and the at least one second light-emitting pixel that emits light in the second direction and the at least one second light-sensing pixel that senses light incident in the second direction may be alternately arranged in the first and second light-detecting areas.

According to the embodiments of the disclosure, a wearable display device and an electronic device using the same can be worn on a user's body in a ring or cylindrical shape like a ring, a bracelet, a watch, or a band, so that it is possible to quickly and accurately measure the user's biometric information such as blood pressure, heart rate and oxygen saturation in real time.

According to the embodiments of the disclosure, a wearable display device and an electronic device using the same can sense a user's motion by emitting lights on both the inner and outer surfaces to sense reflected lights on the both surfaces, as well as detecting the user's biometric information. Accordingly, wearable display devices can find more applications.

It should be noted that effects of the disclosure are not limited to those described above and other effects of the disclosure will be apparent to those skilled in the art from the following descriptions.

### BRIEF DESCRIPTION OF THE DRAWINGS

The above and other aspects and features of the disclosure will become more apparent by describing in detail embodiments thereof with reference to the attached drawings, in which:

FIG. 1 is a perspective view showing a wearable display device according to an embodiment of the disclosure.

FIG. 2 is a schematic cross-sectional view showing a cross-sectional structure of the wearable display device shown in FIG. 1.

FIG. 3 is a top view showing the display panel shown in FIG. 2 that is spread out according to an embodiment.

FIG. 4 is a schematic diagram of an equivalent circuit of a first light-emitting pixel disposed in the biological signal detection area of FIG. 3.

FIG. 5 is a schematic diagram of an equivalent circuit of a first light-sensing pixel disposed in the biological signal detection area of FIG. 3.

FIG. 6 is a schematic cross-sectional view schematically showing the cross-sectional structure taken along line I-I of FIG. 3.

FIG. 7 is a schematic cross-sectional view showing the cross-sectional structure taken along line I-I′ of FIG. 6.

FIG. 8 is a graph for illustrating a method for calculating information about blood pressure among biometric information according to an embodiment.

FIG. 9 is a graph for illustrating a method of calculating information about heart rate and respiration among biometric information according to an embodiment.

FIG. 10 is a schematic cross-sectional view schematically showing the cross-sectional structure of a second light-emitting pixel and a second light-sensing pixel disposed in the touch detection area of FIG. 3.

FIG. 11 is a schematic cross-sectional view showing the cross-sectional structure of the second light-emitting pixel and the second light-sensing pixel shown in FIG. 10.

FIG. 12 is a top view showing the display panel shown in FIG. 2 that is spread out according to an embodiment.

FIG. 13 is a schematic block diagram of an electronic device according to an embodiment.

FIGS. 14, 15 and 16 are schematic diagrams of electronic devices according to various embodiments.

### DETAILED DESCRIPTION OF THE EMBODIMENTS

The disclosure will now be described more fully hereinafter with reference to the accompanying drawings, in which non-limiting embodiments of the disclosure are shown. The disclosure may, however, be embodied in different forms and should not be construed as limited to the described embodiments set forth herein.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. Also, when an element is referred to as being “in contact” or “contacted” or the like to another element, the element may be in “electrical contact” or in “physical contact” with another element; or in “indirect contact” or in “direct contact” with another element.

It will be understood that, although the terms “first,” “second,” etc. may be used herein to describe various elements, these elements should not be limited by these terms. These terms are only used to distinguish one element from another element. For instance, a first element discussed below could be termed a second element without departing from the teachings of the disclosure. Similarly, the second element could also be termed the first element.

In the specification and the claims, the phrase “at least one of” is intended to include the meaning of “at least one selected from the group of” for the purpose of its meaning and interpretation. For example, “at least one of A and B” may be understood to mean “A, B, or A and B.” In the specification and the claims, the term “and/or” is intended to include any combination of the terms “and” and “or” for the purpose of its meaning and interpretation. For example, “A and/or B” may be understood to mean “A, B, or A and B.” The terms “and” and “or” may be used in the conjunctive or disjunctive sense and may be understood to be equivalent to “and/or.”

The term “about” may include variations of, for example, ±20%, ±10%, or ±5%, from the specified numerical value unless otherwise expressly stated. In some contexts, the term may account for rounding, inherent measurement limitations, or standard tolerances recognized in the relevant technical field. When applied to dimensions, concentrations, or other quantifiable parameters, “about” may include minor deviations that would be understood by a person of ordinary skill in the art as insubstantial in the given context. The scope of “about” should be interpreted in view of standard experimental or clinical tolerances applicable to the field of use. A person skilled in the art would recognize that “about” allows for practical deviations that do not materially alter the intended properties of the invention. Similarly, for mechanical dimensions, “about” may include deviations that are within industry-accepted tolerances and do not materially impact the performance of the disclosure.

Each of the features of the various embodiments of the disclosure may be combined or combined with each other, in part or in whole, and technically various interlocking and driving are possible. Each embodiment may be implemented independently of each other or may be implemented together in an association.

Unless otherwise defined or implied herein, all terms (including technical and scientific terms) used have the same meaning as commonly understood by those skilled in the art to which this disclosure pertains. It will be further understood that terms, such as those defined in commonly used dictionaries, should be interpreted as having a meaning that is consistent with their meaning in the context of the relevant art and should not be interpreted in an ideal or excessively formal sense unless clearly defined in the specification.

Hereinafter, embodiments of the disclosure will be described with reference to the accompanying drawings.

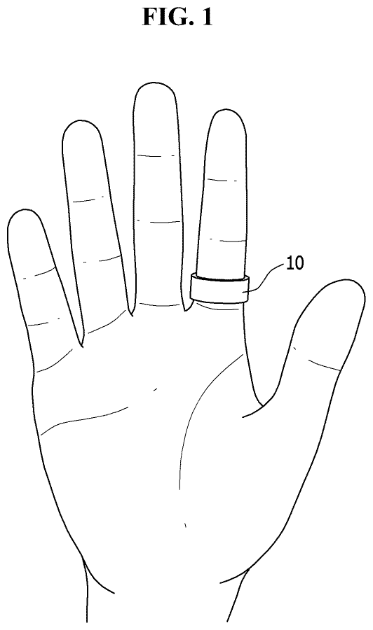

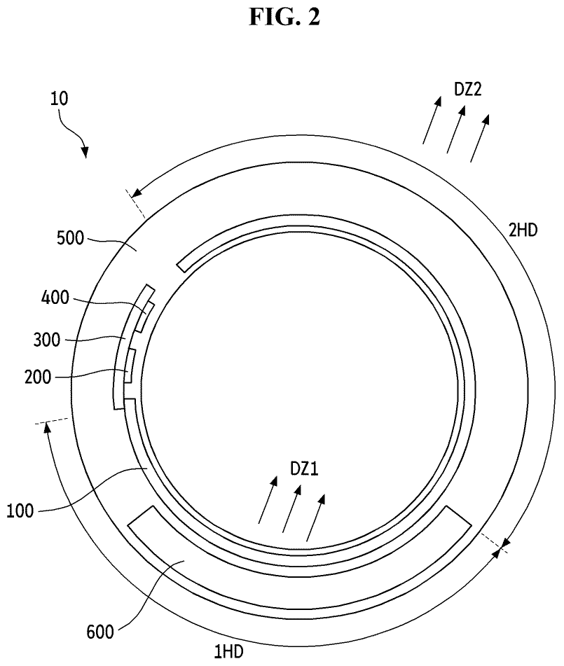

FIG. 1 is a perspective view showing a wearable display device according to an embodiment of the disclosure. FIG. 2 is a schematic cross-sectional view showing a cross-sectional structure of the wearable display device shown in FIG. 1.

Referring to FIGS. 1 and 2, the wearable display device 10 according to the embodiment may be used for electronic devices that are a ring or cylindrical type which can be worn on a user's body, like a ring, a bracelet, a smart watch, a band, a watch phone, etc.

The wearable display device 10 may include a light-emitting display device such as an organic light-emitting display device using organic light-emitting diodes, a quantum-dot light-emitting display device including quantum-dot light-emitting layer, an inorganic light-emitting display device including an inorganic semiconductor, and a micro-LED display device using micro or nano light-emitting diodes (micro LEDs or nano LEDs) depending on the emission structure. In the following description, an organic light-emitting structure using an organic light-emitting diode is employed as the light-emitting structure of the wearable display device 10. It should be understood, however, that the disclosure is not limited thereto.

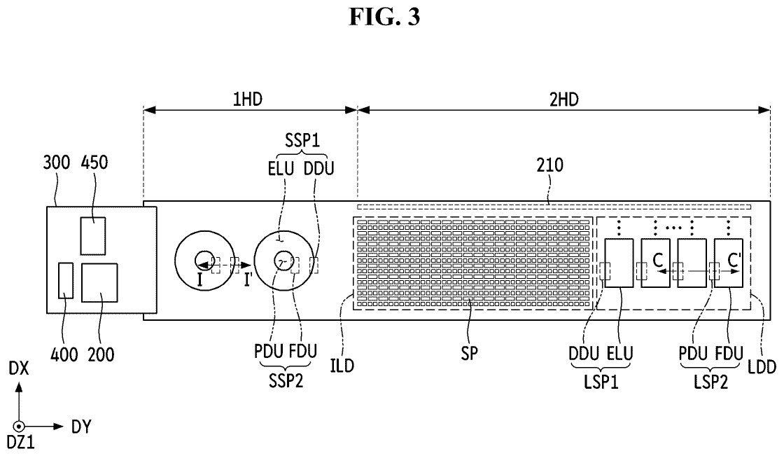

FIG. 3 is a top view showing the display panel shown in FIG. 2 that is spread out according to an embodiment.

Referring to FIGS. 2 and 3, the wearable display device 10 according to an embodiment may include a display panel 100, a main driver circuit 200, a scan driver 210, a circuit board 300, a sensing circuit 450, a memory 400, and a housing 500.

The housing 500 may be formed in a ring or cylindrical shape that may be applied to a ring, a bracelet, a watch, a band, etc. Multiple grooves in which the display panel 100, the main driver circuit 200, the circuit board 300, etc., are embedded may be formed on the inner surface and the inside of the housing 500. The housing 500 may be made of an opaque plastic, rubber, silicone, or a metal material. Transparent glass or plastic may be formed in an area corresponding to the emission areas or light-receiving areas of the display panel 100.

The display panel 100 may be disposed in a ring shape or a cylindrical shape inside the housing 500. The display panel 100 may emit light and receive light in a first direction DZ1, i.e., toward the inside of the cylindrical housing 500 to detect a first light-sensing signal according to the amount of light reflected from the user's body. The display panel 100 may also emit light and receive light in a second direction DZ2, i.e., toward the outside of the cylindrical housing 500 to detect a second light-sensing signal according to the amount of light reflected from the user's body.

The main driver circuit 200 may detect a user's pulse wave signal and measure the user's biometric information using the first light-sensing signal of the display panel 100, and may sense the movement of a touch by a part of the user's body using the second light-sensing signal. For example, the main driver circuit 200 may receive first light-sensing signals generated in the display panel 100 through light-sensing lines formed in the display panel 100, and may detect photoplethysmography signals, i.e., pulse wave signals, among the biological signals proportional to changes in the magnitude of the first light-sensing signals.

The main driver circuit 200 may analyze pulse wave signals every predetermined period and measure biometric information such as blood pressure, heart rate, heart rate variability, respiratory rate, blood vessel elasticity, cardiovascular disease, and oxygen saturation. The main driver circuit 200 may store the biometric information in the memory 400 and may transmit the biometric information stored in the memory 400 to another mobile display device, etc. through a separate cable, a communication circuit, an antenna, etc. The biometric information measurements such as blood pressure, heart rate, heart rate variability, respiratory rate, vascular elasticity, cardiovascular disease, and oxygen saturation may be displayed by an application program on the screen of the mobile display device.

The main driver circuit 200 may generate electric signals for driving the display panel 100 such as control signals and data voltages. The main driving circuit 200 may be implemented as an integrated circuit (IC) and may be attached to the display panel 10 or the circuit board 300 by a chip on glass (COG) technique, a chip on plastic (COP) technique, or an ultrasonic bonding. It is, however, to be understood that the disclosure is not limited thereto. For example, the main driver circuit 200 may be attached on the circuit board 300 by a chip-on-film (COF) technique.

The scan driver 210 may sequentially provide gate scan signals to the first and second light-emitting pixels and the first and second light-sensing pixels disposed in the display panel 100, thereby controlling the pixels so that the first and second light-emitting pixels emit lights and the first and second light-sensing pixels receive lights.

The scan driver 210 may receive an emission control signal from the main driver circuit 200, and sequentially generate emission scan signals every horizontal line driving period in response to the emission control signal to sequentially provide emission scan signals to the first and second light-emitting pixels. In other words, the scan driver 210 may sequentially control the emission timing of the first and second light-emitting pixels. The scan driver 210 may sequentially generate light-sensing scan signals in response to a scan control signal from the main driver circuit 200 and sequentially provide light-sensing scan signals to the first and second light-sensing pixels. For example, the scan driver 210 may sequentially control the light-sensing timing of the first and second light-sensing pixels.

The main driver circuit 200 may be mounted on the circuit board 300, and the main driver circuit 200 may be electrically connected to the display panel 100 through the circuit board 300. The circuit board 300 may be attached to an end of the display panel 100. Accordingly, the circuit board 300 may be electrically connected to the display panel 100 and the main driver circuit 200. The circuit board 300 may be a flexible printed circuit board, a printed circuit board, or a flexible film such as a chip-on film.

The sensing circuit 450 may be disposed in the housing 500 or the circuit board 300 and sense the direction of the user's movement and the pressure applied by the user's finger. To this end, the sensing circuit 450 may include a motion detection sensor such as a gyro sensor and a pressure sensor. The sensing circuit 450 may generate a motion detection signal in real time to transmit a motion detection signal to the main driver circuit 200, allowing the main driver circuit 200 to check the user's movement information in real time. The sensing circuit 450 may transmit a pressure detection signal corresponding to a change in pressure applied by the user's finger to the main driving circuit 200.

The memory 400 may store the user's pulse wave signals and the user's biometric information generated in the main driver circuit 200, and allow the user's pulse wave signals and the user's biometric information to be transmitted to other mobile display devices, etc., through a separate cable or a communication circuit, and antenna, etc.

Referring to FIG. 3, the display panel 100 may include a biological signal detection area 1HD and a touch detection area 2HD.

At least one first light-emitting pixel SSP1 and at least one first light-sensing pixel SSP2 may be formed and disposed in the biological signal detection area 1HD.

At least one first light-emitting pixel SSP1 may emit light in the first direction DZ1, i.e., toward the inside of the display panel 100 disposed in a cylindrical shape, e.g., toward the inside of the cylindrical housing 500. The first light-emitting pixel SSP1 may be defined as a light-emitting pixel that is the minimum unit for emitting light of one of red, green, blue and white. For example, at least one first light-emitting pixel SSP1 may be formed as a light-emitting pixel that emits green light used as a reflected light source for the vein of a finger or a wrist, among other body parts of the user.

At least one first light-emitting pixel SSP1 may include a light-emitting unit ELU that emits light in a first wavelength range, and a pixel driving unit (or pixel driver) DDU that applies a driving current to a light-emitting element of the light-emitting unit ELU. In an embodiment, the light in the first wavelength range may be light in a green wavelength range. For example, the main peak wavelength of the light in the first wavelength range may be in a range of about 480 nm to about 560 nm.

The pixel driving unit DDU may apply a driving current to the light-emitting element of the light-emitting unit ELU in response to the data voltage from the main driver circuit 200 and the gate scan signal from the scan driver 210. The light-emitting element of the light-emitting unit ELU may emit light in the green wavelength range by the amount of the driving current applied from the pixel driving unit DDU. The light-emitting element of the light-emitting unit ELU may be formed in a circular shape or a polygonal shape, such as a rectangle in a plan view, and may surround a photo-detecting unit of the first light-sensing pixel SSP2.

At least one first light-sensing pixel SSP2 disposed in the biological signal detection area 1HD may receive light reflected from a part of the user's body in the first direction DZ1 and generate a first light-sensing signal based on the amount of received light. The first light-sensing pixel SSP2 may be defined as a light-sensing pixel that is the minimum unit for outputting an electrical signal corresponding to the amount of received light as a light-sensing signal.

At least one first light-sensing pixel SSP2 may include a photo-detecting unit (or photo-detecting part) PDU that outputs a light-sensing current proportional to the amount of incident light in the first direction DZ1, and a sense driving unit (or sensing driver) FDU that provides a first light-sensing signal proportional to the amount of the light-sensing current from the photo-detecting unit PDU to the main driver circuit 200.

The photo-detecting unit PDU may include a photo-detector PD that generates and outputs a light-sensing current proportional to the amount of received light. The photo-detector PD of the photo-detecting unit PDU may be disposed adjacent to the light-emitting unit ELU of the first light-emitting pixel SSP1. The photo-detector PD may be formed in a circular shape or a polygonal shape, such as a rectangle in a plan view and may be surrounded by the light-emitting unit ELU. The photo-detecting unit PDU may have a transmittance in a range of about 80% to about 100% and may detect light in a low brightness or low-illuminance range. To this end, only a transparent protective layer or protective cover, etc., may be disposed on the front side of the photo-detecting unit PDU. For example, a low brightness range may be set in a range of about 0.0005 cd/m² to about 0.0001 cd/m², and the photo-detecting unit PDU may be formed with a transmittance in a range of about 80% to about 100% to detect light in a low brightness range.

The touch detection area 2HD of the display panel 100 may include an image display area ILD and a light-detecting area LDD.

The image display area ILD may include multiple pixels that emits light in the second direction DZ2 that is the outward direction to display an image such as text. The pixels may be arranged in vertical or horizontal stripes or in a PenTile™ matrix, and display text or images in response to scan signals and data voltages from the main driver circuit 200.

The light-detecting area LDD may be parallel to the image display area ILD in the vertical or horizontal direction.

In the light-detecting area LDD, multiple second light-emitting pixels LSP1 and multiple second light-sensing pixels LSP2 may be alternately arranged in horizontal and vertical stripes. For example, multiple second light-emitting pixels LSP1 and multiple second light-sensing pixels LSP2 may be arranged alternately in a matrix pattern.

The second light-emitting pixels LSP1 may emit light in the second direction DZ2, i.e., in the outward direction of the display panel 100 in a cylindrical shape, e.g., toward the outside of the cylindrical housing 500. Each of the second light-emitting pixels LSP1 may be defined as a light-emitting pixel that is the minimum unit for emitting light of one of red, green, blue and white. For example, the second light-emitting pixels LSP1 may be formed as a light-emitting pixel that emits red or white light used as a reflected light source for the skin of a finger or a palm, among other body parts of the user.

For example, the second light-emitting pixels LSP1 may include a light-emitting unit ELU that emits light in a first wavelength range, and a pixel driving unit DDU that applies a driving current to a light-emitting element of the light-emitting unit ELU. In an embodiment, the light in the first wavelength range may be light in a green wavelength range.

The pixel driving unit DDU may apply a driving current to the light-emitting element of the light-emitting unit ELU in response to the data voltage from the main driver circuit 200 and the gate scan signal from the scan driver 210. The light-emitting element of the light-emitting unit ELU may emit light in a green wavelength range by the amount of the driving current applied from the pixel driving unit DDU. The light-emitting element of the light-emitting unit ELU may have a circle or a polygonal shape such as a rectangle in a plan view.

The second light-sensing pixels LSP2 arranged in the light-detecting area LDD may receive light reflected from a part of the user's body in the second direction DZ2 and generate a second light-sensing signal according to the amount of light received. The second light-sensing pixels LSP2 may be defined as a light-sensing pixel that is the minimum unit for outputting an electrical signal corresponding to the amount of received light as a light-sensing signal.

The second light-sensing pixels LSP2 may include a photo-detecting unit PDU that outputs a light-sensing current proportional to the amount of incident light in the second direction DZ2, and a sense driving unit FDU that provides a second light-sensing signal proportional to the amount of the light-sensing current from the photo-detecting unit PDU to the main driver circuit 200.

The photo-detecting unit PDU may include a photo-detector PD that generates and outputs a light-sensing current proportional to the amount of received light. The photo-detector PD of the photo-detecting unit PDU may be disposed adjacent to the light-emitting unit ELU of the second light-emitting pixel LSP1. The photo-detector PD may have a circle or a polygonal shape such as a rectangle in a plan view. The photo-detecting unit PDU may have a transmittance in a range of about 80% to about 100% and may detect light in a low brightness or low-illuminance range. To this end, only a transparent protective layer or protective cover, etc., may be disposed on the front side of the photo-detecting unit PDU.

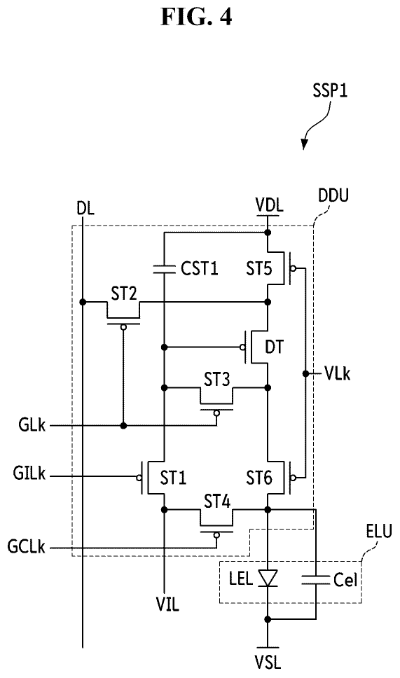

FIG. 4 is a schematic diagram of an equivalent circuit of a first light-emitting pixel disposed in the biological signal detection area of FIG. 3.

Referring to FIG. 4, each of the first light-emitting pixels SSP1 according to an embodiment may be connected to a kth display initialization line GILk, a k^{th} display scan line GLk, a k^{th} display control line GCLk, and a k^{th} emission control line VLK. The first display pixel SPI may be connected to a first supply voltage line VDL from which a first supply voltage is supplied, a second supply voltage line VSL from which a second supply voltage is supplied, and a third supply voltage line VIL from which a third supply voltage is supplied. In the following description, the letters such as k and n used in place of numbers are defined as positive integers excluding zero.

As described above, the first light-emitting pixel SSP1 may include the light-emitting unit ELU and the pixel driving unit DDU. The light-emitting unit ELU may include a light-emitting element LEL. The pixel driving unit DDU may include a driving transistor DT, switch elements, and a capacitor CST1. The switch elements may include first to sixth transistors ST1, ST2, ST3, ST4, ST5 and ST6.

The driving transistor DT may include a gate electrode, a first electrode and a second electrode. A drain-source current Ids (hereinafter referred to as “driving current”) of driving transistor DT flowing between the first electrode and the second electrode may be controlled according to the data voltage applied to the gate electrode. The driving current Ids flowing through the channel of the driving transistor DT may be proportional to the square of the difference of voltages Vgs between the first electrode and the gate electrode of the driving transistor DT and the threshold voltage Vth, as shown in Equation 1 below:

$$Ids = {k}^{′} \times {( Vsg - Vth )}^{2} \qquad [ Equation l ]$$

where k′ may be a proportional coefficient determined by the structure and physical properties of the driving transistor, Vsg may be a difference of voltages between the first electrode and the gate electrode of the driving transistor, and Vth may be a threshold voltage of the driving transistor.

The light-emitting element LEL may emit light as the driving current Ids flows through the light-emitting element LEL. The amount of the light emitted from the light-emitting elements LEL may increase with the driving current Ids.

The light-emitting element LEL may be an organic light-emitting diode including an organic emissive layer disposed between an anode electrode and a cathode electrode. In another embodiment, the light-emitting element LEL may be an inorganic light-emitting element including an inorganic semiconductor disposed between an anode electrode and a cathode electrode. In another embodiment, the light-emitting element LEL may be quantum-dot light-emitting element including a quantum-dot emissive layer disposed between an anode electrode and a cathode electrode. In another embodiment, the light-emitting element LEL may be a micro light-emitting element including a micro light-emitting diode disposed between an anode electrode and a cathode electrode.

The anode electrode of the light-emitting element LEL may be connected to the first electrode of the fourth transistor ST4 and the second electrode of the sixth transistor ST6, while the cathode electrode of the light-emitting element LEL may be connected to the second supply voltage line VSL. A parasitic capacitor Cel may be formed between the anode electrode and the cathode electrode of the light-emitting element LEL.

The first transistor ST1 may be turned on by an initialization scan initialization signal of the k^{th} display initialization line GILk to connect the gate electrode of the driving transistor DT with the third supply voltage line VIL1. Accordingly, a third supply voltage of the third supply voltage line VIL1 may be applied to the gate electrode of the driving transistor DT. The gate electrode of the first transistor ST1 may be connected to the k^{th} display initialization line GILk, the first electrode of the first transistor ST1 may be connected to the gate electrode of the driving transistor DT, and the second electrode of the first transistor ST1 may be connected to the third driving voltage line VIL.

The second transistor ST2 may be turned on by the display scan signal of the k^{th} display scan line GLk to connect the first electrode of the driving transistor DT with the data line DL. Accordingly, the data voltage of the data line DL may be applied to the first electrode of the driving transistor DT. The gate electrode of the second transistor ST2 may be connected to the k^{th} display scan line GLk, a first electrode of the second transistor ST2 may be connected to the first electrode of the driving transistor DT, and a second electrode of the second transistor ST2 may be connected to the data line DL.

The third transistor ST3 may be turned on by the display scan signal of the k^{th} display scan line GLk to connect the gate electrode and the second electrode of the driving transistor DT with each other. In case that the gate electrode and the second electrode of the driving transistor DT are connected with each other, the driving transistor DT may work as a diode. A gate electrode of the third transistor ST3 may be connected to the k^{th} display scan line GLk, a first electrode of the third transistor ST3 may be connected to the second electrode of the driving transistor DT, and a second electrode of the third transistor ST3 may be connected to the gate electrode of the driving transistor DT.

The fourth transistor ST4 may be turned on by a display control signal of the k^{th} display control line GCLk to connect the anode electrode of the light-emitting element LEL with the third supply voltage line VIL. The third supply voltage of the third supply voltage line VIL may be applied to the anode electrode of the light-emitting element LEL. The gate electrode of the fourth transistor ST4 may be connected to the k^{th} display control line GCLk, the first electrode of the fourth transistor ST4 may be connected to the anode electrode of the light-emitting element LEL, and the second electrode of the fourth transistor ST4 may be connected to the third supply voltage line VIL.

The fifth transistor ST5 may be turned on by the emission signal of a k^{th} emission control line VLK to connect the first electrode of the driving transistor DT with the first supply voltage line VDL. The gate electrode of the fifth transistor ST5 may be connected to the k^{th} emission control line VLK, the first electrode of the fifth transistor ST5 may be connected to the first supply voltage line VDL, and the second electrode of the fifth transistor ST5 may be connected to the first electrode of the driving transistor DT.

The sixth transistor ST6 may be disposed between the second electrode of the driving transistor DT and the anode electrode of the light-emitting element LEL. The sixth transistor ST6 may be turned on by the emission control signal of the k^{th} emission control line VLk to connect the second electrode of the driving transistor DT with the anode electrode of the light-emitting element LEL. The gate electrode of the sixth transistor ST6 may be connected to the k^{th} emission control line VLK, the first electrode of the sixth transistor ST6 may be connected to the second electrode of the driving transistor DT, and the second electrode of the sixth transistor ST6 may be connected to the anode electrode of the light-emitting element LEL.

In case that both the fifth transistor ST5 and the sixth transistor ST6 are turned on, the driving current of the driving transistor DT according to the data voltage applied the gate electrode of the driving transistor DT may flow to the light-emitting element LEL.

The capacitor CST1 may be formed between the gate electrode of the driving transistor DT and the first supply voltage line VDL. The first capacitor electrode of the capacitor CST1 may be connected to the gate electrode of the driving transistor DT, and the second capacitor electrode of the capacitor CST1 may be connected to the first driving voltage line VDL.

In case that the first electrode of each of the first to sixth transistors ST1, ST2, ST3, ST4, ST5 and ST6 and the driving transistor DT is a source electrode, the second electrode of each of the first to sixth transistors ST1, ST2, ST3, ST4, ST5 and ST6 may be a drain electrode. In case that the first electrode of each of the first to sixth transistors ST1, ST2, ST3, ST4, ST5 and ST6 and the driving transistor DT is a drain electrode, the second electrode of each of the first to sixth transistors ST1, ST2, ST3, ST4, ST5 and ST6 may be a source electrode.

The active layer of each of the first to sixth transistors ST1, ST2, ST3, ST4, ST5 and ST6 and the driving transistor DT may be formed of one of poly silicon, amorphous silicon and oxide semiconductor. Although the first to sixth transistors ST1, ST2, ST3, ST4, ST5 and ST6 and the driving transistor DT are implemented as p-type metal oxide semiconductor field effect transistors (MOSFETs) in FIG. 4, the disclosure is not limited thereto. For example, the first to sixth transistors ST1, ST2, ST3, ST4, ST5 and ST6, and the driving transistor DT may be implemented as n-type MOSFETs. For example, at least one of the first to sixth transistors ST1, ST2, ST3, ST4, ST5 and ST6 may be implemented as an n-type MOSFET.

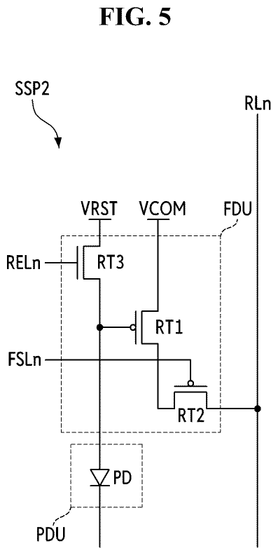

FIG. 5 is a schematic diagram of an equivalent circuit of a first light-sensing pixel disposed in the biological signal detection area of FIG. 3.

Referring to FIG. 5, the first light-sensing pixel SSP2 may be electrically connected to an n^{th} sensing reset line RELn, an n^{th} light-sensing scan line FSLn, and an n^{th} light-sensing line RLn. Each of the first light-sensing pixels SSP2 may be reset by a reset signal from the n^{th} sensing reset line RELn, and may transmit a light-sensing signal to the n^{th} light-sensing line RLn in response to the sensing scan signal from the n^{th} light-sensing scan line FSLn.

The first light-sensing pixels SSP2 may be divided into a photo-detecting unit PDU including a photo-detecting element PD, and a sense driving unit FDU including first to third sensing transistors RT1 to RT3 and a sensing capacitor (not shown). The sensing capacitor may be formed in parallel with the photo-detecting elements PD.

A first sensing transistor RT1 of the sense driving unit FDU may allow a light-sensing current to flow according to the voltages of the photo-detecting element PD and the sensing capacitor. The amount of current of the light-sensing current may depend on a voltage applied to the photo-detecting element PD and the sensing capacitor. The gate electrode of the first sensing transistor RT1 may be connected to the second electrode of the photo-detecting element PD. A first electrode of the first sensing transistor RT1 may be connected to a common voltage source VCOM from which a common voltage is applied. A second electrode of the first sensing transistor RT1 may be connected to a first electrode of the second sensing transistor RT2.

In case that the sensing scan signal of the gate-on voltage is applied to the n^{th} light-sensing scan line FSLn, the second sensing transistor RT2 may allow the sensing current of the first sensing transistor RT1 to flow to the n^{th} light-sensing line RLn. In this instance, the n^{th} light-sensing line RLn may be charged with the sensing voltage by the sensing current. The gate electrode of the second sensing transistor RT2 may be connected to the n^{th} light-sensing scan line FSLn, the first electrode of the second sensing transistor RT2 may be connected to the second electrode of the first sensing transistor RT1, and the second electrode of the second sensing transistor RT2 may be connected to the n^{th} light-sensing line RLn.

In case that a reset signal of the gate-on voltage is applied to the n^{th} sensing reset line RELn, the third sensing transistor RT3 may reset the voltages of the photo-detecting element PD and the sensing capacitor to the reset voltage of a reset voltage source VRST. The gate electrode of the third sensing transistor RT3 may be connected to the sensing reset line RELn, the first electrode of the third sensing transistor RT3 may be connected to the reset voltage source VRST, and the second electrode of the third sensing transistor RT3 may be connected to the second electrode of the photo-detecting element PD.

Although the first sensing transistor RT1 and the second sensing transistor RT2 are implemented as p-type metal oxide semiconductor field effect transistors (MOSFETs) while the third sensing transistor RT3 is implemented as an n-type MOSFET in the embodiment shown in FIG. 5, the disclosure is not limited thereto. In another embodiment, the first sensing transistor RT1, the second sensing transistor RT2, and the third sensing transistor RT3 may be of a same type or different types. One of the first and second electrodes of each of the first sensing transistor RT1, the second sensing transistor RT2 and the third sensing transistor RT3 may be a source electrode, while another one of the first and second electrodes of each of the first sensing transistor RT1, the second sensing transistor RT2 and the third sensing transistor RT3 may be a drain electrode.

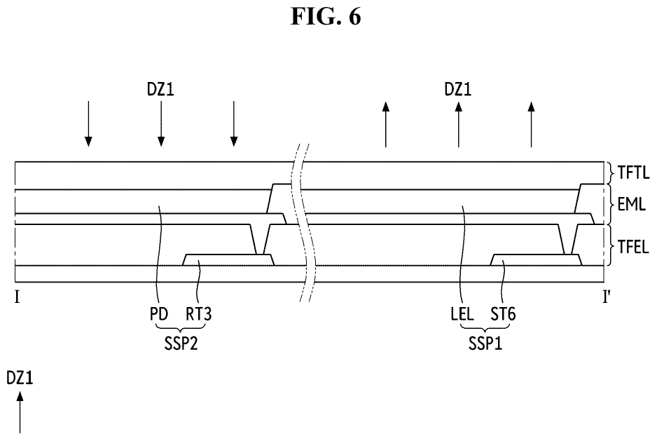

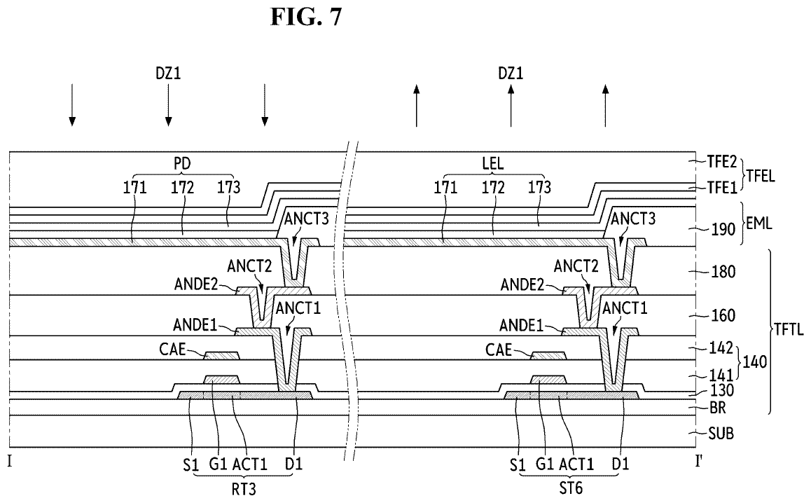

FIG. 6 is a schematic cross-sectional view schematically showing the cross-sectional structure taken along line I-I of FIG. 3. FIG. 7 is a schematic cross-sectional view showing the cross-sectional structure taken along line I-I′ of FIG. 6.

First, referring to FIGS. 3 and 6, a photo-detector PD of a photo-detecting unit PDU formed in a first light-sensing pixel SSP2 may be formed in a circular shape or a polygonal shape such as a rectangle in a plan view, and may be surrounded by a light-emitting unit ELU.

The light-emitting elements LEL formed in the light-emitting unit ELU of the first light-emitting pixel SSP1 may be formed in a circular shape or a polygonal shape such as a rectangle in a plan view, and may surround the photo-detecting unit PDU of the first light-sensing pixel SSP2.

Referring to FIGS. 6 and 7, the substrate SUB of the display panel 100 may be a base substrate or a base member. The substrate SUB may be a flexible substrate that can be bent, folded, or rolled. The substrate SUB may include a polymer resin including polyimide PI.

The thin-film transistor layer TFTL may be disposed on the substrate SUB. The thin-film transistor layer TFTL may include multiple thin-film transistors that form a pixel driving unit DDU of the first light-emitting pixel SSP1 and a sense driving unit FDU of the first light-sensing pixel SSP2. The thin-film transistor layer TFEL may include gate lines, data lines, voltage lines, gate control lines, fan-out lines for connecting the main driver circuit 200 with the data lines, lead lines for connecting the main driver circuit 200 with the pads, etc. In case that the scan driver 210 is formed on an end of the display panel 100, the scan driver 210 may include thin-film transistors.

The emission layer EML may be disposed on the thin-film transistor layer TFEL. The emission layer EML may include multiple light-emitting elements in each of which a first electrode, an organic material layer and a second electrode are stacked on one another sequentially to emit light, and a pixel-defining layer for defining an area in which each of light-emitting elements PD is formed.

An encapsulation layer TFTL may cover the upper and side surfaces of the emission layer EML, and may protect the emission layer EML. The encapsulation layer TFTL may include at least one inorganic layer and at least one organic layer for encapsulating the emission layer EML.

Referring to FIG. 7, the barrier layer BR may be a layer for protecting the transistors of the thin-film transistor layer TFTL and an organic material layer 172 of the emission layer EML. The barrier layer BR may be made up of multiple inorganic layers alternately stacked on one another.

The thin-film transistor ST6 and RT3, etc., may be disposed on the barrier layer BR. Each of the thin-film transistors ST6 and RT3 may include an active layer ACT1, a gate electrode G1, a source electrode S1 and a drain electrode D1.

The active layer ACT1, the source electrode S1 and the drain electrode D1 of each of the thin-film transistors ST6, RT3 may be disposed on the barrier layer BR. The active layer ACT1 of each of the thin-film transistors ST6 and RT3 may include polycrystalline silicon, single crystalline silicon, low-temperature polycrystalline silicon, amorphous silicon, or an oxide semiconductor. A part of the active layer ACT1 overlapping the gate electrode G1 in the third direction (z-axis direction DZ1) that is the thickness direction of the substrate SUB may be defined as a channel region. The source electrode S1 and the drain electrode D1 may be regions that do not overlap the gate electrode G1 in the third direction (z-axis direction DZ1), and may have conductivity by doping ions or impurities into a silicon semiconductor or an oxide semiconductor.

A gate insulator 130 may be disposed on the active layer ACT1, the source electrode S1 and the drain electrode D1 of each of the thin-film transistors ST6 and RT3.

The gate electrode G1 of each of the thin-film transistors ST6 and RT3 may be disposed on the gate insulator 130. The gate electrode G1 may overlap the active layer ACT1 in the third direction (z-axis direction DZ1).

A first interlayer dielectric layer 141 may be disposed on the gate electrode G1 of each of the thin-film transistors ST1.

A capacitor electrode CAE may be disposed on the first interlayer dielectric layer 141. The capacitor electrode CAE may overlap the gate electrode G1 of each of the thin-film transistors ST6 and RT in the third direction (z-axis direction DZ1). Since the first interlayer dielectric layer 141 has a dielectric constant, a capacitor may be formed by the capacitor electrode CAE, the gate electrode G1, and the first interlayer dielectric layer 141 disposed between the capacitor electrode CAE and the gate electrode G1. A second interlayer dielectric layer 142 may be disposed over the capacitor electrode CAE.

A first anode connection electrode ANDE1 may be disposed on the second interlayer dielectric layer 142. The first anode connection electrode ANDE1 may be connected to the drain electrode D1 of each of the thin-film transistors ST6 and RT3 through a first connection contact hole ANCT1 that penetrates the gate insulator 130, the first interlayer dielectric layer 141 and the second interlayer dielectric layer 142. The first anode connection electrode ANDE1 may be made up of a single layer or multiple layers including at least one of molybdenum (Mo), aluminum (Al), chromium (Cr), gold (Au), titanium (Ti), nickel (Ni), neodymium (Nd), copper (Cu), and an alloy thereof. A first planarization layer 160 may be disposed over the first anode connection electrode ANDE1 for providing a flat surface over the thin-film transistors ST6 and RT3.

A second anode connection electrode ANDE2 may be disposed on the first planarization layer 160. The second anode connection electrode ANDE2 may be connected to the first anode connection electrode ANDE1 through a second connection contact hole ANCT2 penetrating the first planarization layer 160. The second anode connection electrode ANDE2 may be made up of a single layer or multiple layers including at least one of molybdenum (Mo), aluminum (Al), chromium (Cr), gold (Au), titanium (Ti), nickel (Ni), neodymium (Nd), copper (Cu), and an alloy thereof. A second planarization layer 180 may be disposed on the second anode connection electrode ANDE2.

Light-emitting elements LEL, light-sensing elements PD and a pixel-defining layer 190 may be disposed on the second planarization layer 180. Each of the light-emitting elements LEL and the light-sensing elements PD may include a pixel electrode 171, an organic material layer 172, and a common electrode 173. An organic material made of an organic light-emitting material may be formed in the organic material layer 172 of the light-emitting elements LEL. In the emission area of the light-emitting elements LEL, the pixel electrode 171, the organic material layer 172 and the common electrode 173 may be stacked on one another sequentially, so that holes from the pixel electrode 171 and electrons from the common electrode 173 may be combined with each other in the organic material layer 172 to emit light.

The organic material layer 172 of the light-emitting element LEL formed in the light-emitting unit ELU of the first light-emitting pixel SSP1 may be formed in a circular shape or a polygonal shape such as a rectangle in a plan view, and may surround the photo-detector PD of the first light-sensing pixel SSP2. On the other hand, the organic material layer 172 of the photo-detector PD formed in the first light-sensing pixel SSP2 may be formed in a circular shape or a polygonal shape such as a rectangle in a plan view, and may be surrounded by the light-emitting element LEL.

As described above, the first light-emitting pixel SSP1 of the biological signal detection area 1HD may emit light in the first direction DZ1 which is the inward direction of the display panel 100, and at least one first light-sensing pixel SSP2 may receive light reflected in the first direction DZ1. The structure in which light is emitted in the first direction DZ1, which is the inward direction of the display panel 100, may be defined as a top-emission structure in which light is emitted from the organic material layer 172 toward the common electrode 173.

In a top-emission structure in which light exits from the organic material layer 172 through the common electrode 173, the pixel electrode 171 may be made of a metal material having a high reflectivity such as a stack structure of aluminum and titanium (Ti/Al/Ti), a stack structure of aluminum and indium tin oxide (ITO) (ITO/Al/ITO), an APC alloy and a stack structure of APC alloy and ITO (ITO/APC/ITO). The APC alloy may be an alloy of silver (Ag), palladium (Pd) and copper (Cu).

In a top-emission organic light-emitting diode, the common electrode 173 may be formed of a transparent conductive material (TCP) such as ITO and IZO that can transmit light, or a semi-transmissive conductive material such as magnesium (Mg), silver (Ag) and an alloy of magnesium (Mg) and silver (Ag). In case that the common electrode 173 is formed of a semi-transmissive metal material, the light extraction efficiency may be increased by forming microcavities.

The encapsulation layer TFTL may be disposed on the common electrode 173. The encapsulation layer TFTL may include at least one inorganic layer to prevent permeation of oxygen or moisture into the emission layer EML. The encapsulation layer TFTL may include at least one organic layer to protect the light-emitting element layer EML from foreign substances such as dust. For example, the encapsulation layer TFEL may include a first inorganic encapsulation layer TFE1 and an organic encapsulation layer TFE2. The first inorganic encapsulation layer TFE1 may be disposed on the common electrode 173, and the organic encapsulation layer TFE2 may be disposed on the first inorganic encapsulation layer TFE1.

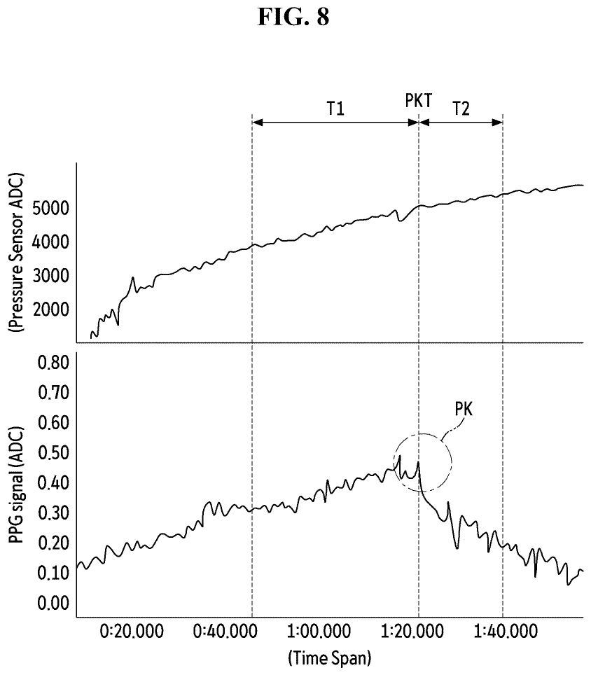

FIG. 8 is a graph for illustrating a method for calculating information about blood pressure among biometric information according to an embodiment.

Referring to FIG. 8, the blood ejected from the left ventricle of a heart a systole of a heart may move to the peripheral tissues, and accordingly the blood volume in the artery may increase. Red blood cells may carry more oxygen in hemoglobin to the peripheral tissues during the systole of the heart. During a diastole of the heart, a part of the blood may be sucked from the peripheral tissues towards the heart. In case that a peripheral blood vessel is irradiated with light, the irradiated light may be absorbed by the peripheral tissue. The light absorbance may be dependent on the hematocrit ratio and the blood volume. The light absorbance may have a maximum value during the systole of the heart and a minimum value during the diastole of the heart. The light absorbance may have a maximum value during the systole of the heart and a minimum value during the diastole of the heart.

In case that a user wears the wearable display device 10 in a blood pressure measurement mode, i.e., in case that the user brings her/his finger into contact with the biological signal detection area 1HD, the pressure (contact pressure) applied to the pressure sensor of the sensing circuit 450 may gradually increase to reach the maximum value and may gradually decrease. As the contact pressure increases, the blood vessels may constrict, resulting in small or zero blood flow rate. In case that the contact pressure decreases, the blood vessels may dilate and blood may begin to flow again. In case that the contact pressure further decreases, the blood flow rate may increase more. Therefore, a change in the amount of light sensed by the first light-sensing pixel SSP2 may be proportional to a change in blood flow. Accordingly, the main driver circuit 200 may generate pulse wave signals PPG according to the pressure applied by the user based on the pressure data value that is calculated by the pressure sensor and digitally converted (ADC of the pressure sensing unit) and the optical signal (PPG signal ratio) according to the amount of light sensed by the photo-detector PD. The pulse wave signal PPG may have a waveform that oscillates according to heartbeat cycle.

The main driver circuit 200 may estimate blood pressures of the blood vessels of the finger based on time differences between time points PKT corresponding to the peaks PK of the calculated pulse wave signals PPG and time points corresponding to the peaks of the filtered pulse waves. For example, the main driver circuit 200 may calculate pulse wave signals for time periods T1 and T2 before and after the time points PKT corresponding to the peaks PK of the calculated pulse wave signals and may detect the blood pressure according to differences between the pulse wave signals. Among the estimated blood pressures, the highest blood pressure may be calculated as the systolic blood pressure while the lowest blood pressure may be calculated as the diastolic blood pressure. Other blood pressures such as the average blood pressure may be calculated using the estimated blood pressures.

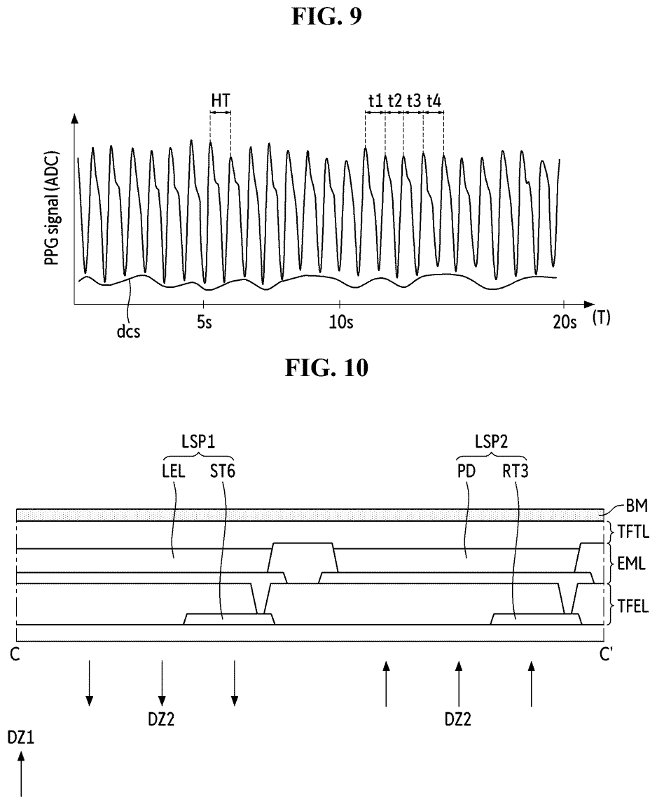

FIG. 9 is a graph for illustrating a method of calculating information about heart rate and respiration among biometric information according to an embodiment.

Referring to FIG. 9, the main driver circuit 200 may set an initial value or reference value for each of the pulse width (e.g., contraction and relaxation cycle), an amplitude (e.g., systolic blood pressure), a systolic pulse width (e.g., the pulse wave signals (PPG)), and an initial value or reference value for each systolic period (e.g., systolic period) and diastolic period (e.g., diastolic pulse width). Subsequently, the main driver circuit 200 may detect changes in the characteristics of pulse wave signals PPG input in real time compared to the initial values or reference values for the characteristics, i.e., changes in the systolic and diastolic period, the systolic blood pressure, the systolic pulse and the diastolic period, and build a database as learning data. The main driver circuit 200 may output information on the blood pressure BP according to change rate or change size of the characteristics of the pulse wave signals PPG compared to the initial values or reference values of the characteristics.

The above-described method for measuring blood pressure is merely illustrative. A variety of methods may be employed such as ones disclosed in Korean Patent Laid-Open Publication Nos. 10-2018-0076050, 10-2017-0049280 and 10-2019-0040527, the entire contents of which are incorporated herein by reference.

The main driver circuit 200 may sample pulse wave signals during a sampling period before and after the time points PKT corresponding to the peaks PK of the pulse wave signals, and may detect generation period HT of high pulses for the sampled pulse wave signals PPG. The main driver circuit 200 may count the number of high pulses for each reference period (e.g., 60 seconds) for the sampled pulse wave signals PPG to detect biometric information about the heart period and heart rate (HR).

The main driver circuit 200 may detect the heart period HT and heart period changes t1 to t4 of the high pulses for each reference period for the peaks PK of the pulse wave signals, to detect heart rate variability (HRV) according to the rate of change of the heart period.

On the other hand, the main driver circuit 200 may sequentially detect the generation period of low pulses and the magnitudes of the low pulses for the sampled pulse wave signals PPG. The main driver circuit 200 may detect the change period of the magnitudes des of the low pulses every predetermined reference period (e.g., 60 seconds) to detect changes in the respiration and respiratory rate (RR) of the user. In doing so, the main driver circuit 200 may analyze a period in which the magnitude des of low pulses rises and a period in which the magnitude des of low pulses falls, to detect the changes in the respiration and the respiratory rate (RR) of the user based on the periods in which the magnitude des of low pulses rises and falls.

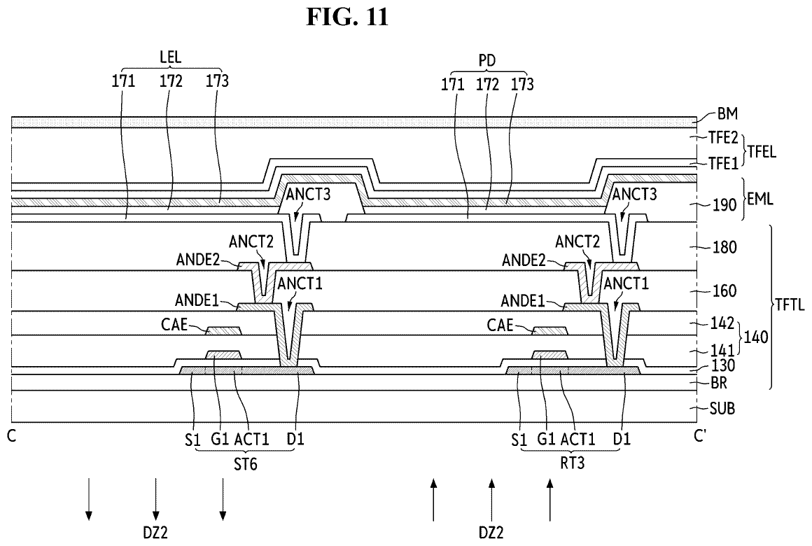

FIG. 10 is a schematic cross-sectional view schematically showing the cross-sectional structure of a second light-emitting pixel and a second light-sensing pixel disposed in the touch detection area of FIG. 3. FIG. 11 is a schematic cross-sectional view showing the cross-sectional structure of the second light-emitting pixel and the second light-sensing pixel shown in FIG. 10.

Referring to FIGS. 10 and 11 in conjunction with FIG. 3, in the touch detection area 2HD of the display panel 100, multiple second light-emitting pixels LSP1 and multiple second light-sensing pixels LSP2 may be arranged alternately in a matrix pattern in the light-detecting area LDD. In another embodiment, multiple second light-emitting pixels LSP1 and multiple second light-sensing pixels LSP2 may be arranged in a PenTile™ matrix in the light-detecting area LDD.

The light-emitting elements LEL of the respective second light-emitting pixels LSP1 arranged in a matrix in the light-detecting area LDD of the touch detection area 2HD may emit light toward the outside of the display panel 100 in a cylindrical shape, e.g., in the second direction DZ2 toward the outside of the cylindrical housing 500. On the other hand, the light-sensing elements PD formed in the second light-sensing pixels LSP2 may detect the amount of light reflected from a part of the user's body in the second direction DZ2 and generate a second light-sensing signal based on the detected amount of light.

As described above, the second light-emitting pixels LSP1 arranged in the light-detecting area LDD may include a light-emitting unit ELU that emits light in a first wavelength range, and a pixel driving unit DDU that applies a driving current to a light-emitting element of the light-emitting unit ELU. The detailed structures of the light-emitting unit ELU and the pixel driving unit DDU of each of the second light-emitting pixels LSP1 is identical to those of the light-emitting unit ELU and the pixel driving unit DDU of the first light-emitting pixel SSP1; and, therefore, the redundant descriptions will be omitted.

The second light-sensing pixels LSP2 arranged in the light-detecting area LDD may include a photo-detecting unit PDU that outputs a light-sensing current proportional to the amount of incident light in the second direction DZ2, and a sense driving unit FDU that provides a second light-sensing signal proportional to the amount of the light-sensing current from the photo-detecting unit PDU to the main driver circuit 200. The detailed structures of the photo-detecting unit PDU and the sense driving unit FDU of each of the second light-sensing pixels LSP2 is identical to those of the light-emitting unit ELU and the pixel driving unit DDU of the first light-emitting pixel SSP2; and, therefore, the redundant descriptions will be omitted.

Referring to FIGS. 10 and 11, the substrate SUB of the display panel 100 may be a base substrate or a base member formed of a transparent silicone, etc. For example, the base substrate in the light-detecting area LDD of the touch detection area 2HD may be a flexible substrate formed of transparent glass, transparent layer, silicone material, etc.

The thin-film transistor layer TFTL may be formed on the substrate SUB. The thin-film transistor layer TFTL may include multiple thin-film transistors that form a pixel driving unit DDU of the second light-emitting pixel LSP1 and a sense driving unit FDU of a second light-sensing pixel LSP2.

The emission layer EML may be disposed on the thin-film transistor layer TFEL. In the emission layer EML, light-emitting elements LEL, photo-detectors PD, and a pixel-defining layer 190 may be formed and arranged. The light-emitting elements LEL and the photo-detector PD may be formed by stacking a first electrode, an organic layer, and a second electrode sequentially.

As described above, the second light-emitting pixels LSP1 disposed in the light-detecting area LDD of the touch detection area 2HD may emit light in the second direction DZ2 which is the outward direction or the rear side direction of the display panel 100 in a cylindrical shape, and the second light-sensing pixels LSP2 may receive light reflected in the second direction DZ2. The structure in which light is emitted in the second direction DZ2, which is the outward direction of the display panel 100, may be defined as a bottom-emission structure in which light is emitted from the organic material layer 172 toward the pixel electrode 171.

In a bottom-emission structure in which light exits from the organic material layer 172 toward the pixel electrode 171, the common electrode 173 may be made of a metal material having a high reflectivity (a metal material that reflects light) such as a stack structure of aluminum and titanium (Ti/Al/Ti), a stack structure of aluminum and indium tin oxide (ITO) (ITO/Al/ITO), an APC alloy and a stack structure of APC alloy and ITO (ITO/APC/ITO). The APC alloy may be an alloy of silver (Ag), palladium (Pd) and copper (Cu).

In a bottom-emission structure, the pixel electrode 171 may be formed of a transparent conductive material (TCP) such as ITO and IZO that can transmit light, or a semi-transmissive conductive material such as magnesium (Mg), silver (Ag) and an alloy of magnesium (Mg) and silver (Ag). In case that the pixel electrode 171 is formed of a semi-transmissive metal material, the light extraction efficiency may be increased by forming microcavities.

An encapsulation layer TFEL may cover the upper and side surfaces of the emission layer EML, and may protect the emission layer EML. The encapsulation layer TFEL may include at least one inorganic layer and at least one organic layer in order to prevent permeation of oxygen or moisture into the emission layer EML.

A black matrix layer BM that can block light may be further formed on the front side of the encapsulation layer TFEL in the light-detecting area LDD of the touch detection area 2HD. The black matrix layer BM of the light-detecting area LDD may block light from being incident in the first direction DZ1 while preventing light from outputting in the first direction DZ1, thereby increasing the first light-sensing efficiency of the light-detecting area LDD.

In the light-detecting area LDD of the touch detection area 2HD, multiple second light-emitting pixels LSP1 and multiple second light-sensing pixels LSP2 may be alternately arranged side-by-side in horizontal and vertical stripes, or may be arranged in a PenTile™ matrix.

Accordingly, the second light-sensing pixels LSP2 arranged in a matrix may generate second light-sensing signals according to the amount of light sensed during a light sensing period, and transmit all of the second light-sensing signals to the main driver circuit 200.

The main driver circuit 200 may receive the second light-sensing signals from the second light-sensing pixels LSP2 arranged in the matrix in the light-detecting area LDD, perform AD conversion (analog-to-digital signal conversion), and store the second light-sensing signals in a memory in a matrix structure. Accordingly, the main driver circuit 200 may analyze and track a change in the magnitude of the second light-sensing signals in the matrix in real time to determine the direction of the user's touch movement.

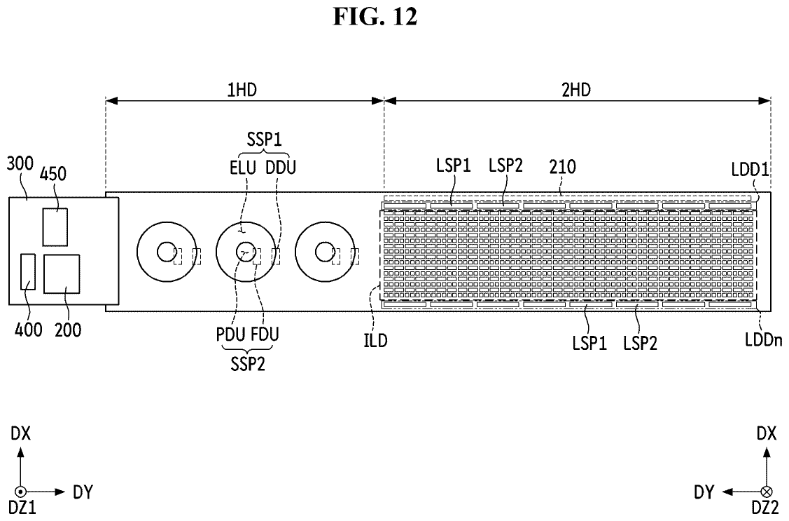

FIG. 12 is a top view showing the display panel shown in FIG. 2 that is spread out according to an embodiment.

Referring to FIG. 12, at least one first light-emitting pixel SSP1 and at least one first light-sensing pixel SSP2 may be formed and disposed in the biological signal detection area 1HD.

On the other hand, the touch detection area 2HD of the display panel 100 may include an image display area ILD, and first to n^{th} light-detecting areas LDD1 and LDDn arranged around the image display area ILD.

The first light-detecting area LDD1 may be located on a side of the image display area ILD. In the first light-detecting area LDD1, second light-emitting pixels LSP1 that emit light in the second direction DZ2, and second light-sensing pixels LSP2 that sense light incident in the second direction DZ2 may be alternately arranged. The second light-emitting pixels LSP1 and the second light-sensing pixels LSP2 alternately formed in the first light-detecting area LDD1 may be arranged side-by-side on a side of the image display area ILD along the longitudinal direction of the display panel 100 and the image display area ILD.

The n^{th} light-detecting area LDDn may be located on an opposite side of the image display area ILD. In the n^{th} light-detecting area LDDn, second light-emitting pixels LSP1 that emit light in the second direction DZ2, and second light-sensing pixels LSP2 that sense light incident in the second direction DZ2 may be alternately arranged. The second light-emitting pixels LSP1 and the second light-sensing pixels LSP2 alternately formed in the n^{th} light-detecting area LDDn may be arranged side-by-side on the opposite side of the image display area ILD along the longitudinal direction of the display panel 100 and the image display area ILD.

Referring to FIG. 12, multiple second light-sensing pixels LSP2 formed in the first light-detecting area LDD1 and second light-sensing pixels LSP2 formed in the n^{th} light-detecting area LDDn may be arranged parallel to each other with the image display area ILD interposed between the first light-detecting area LDD1 and the n^{th} light-detecting area LDDn.

The main driver circuit 200 may receive second light-sensing signals from the second light-sensing pixels LSP2 arranged side-by-side in the first light-detecting area LDD1 and the n^{th} light-detecting area LDDn.

The main driver circuit 200 may determine the direction in which the user's touch moves based on changes in the magnitudes of the second light-sensing signals that are sequentially input from the second light-sensing pixels LSP2 formed in the first light-detecting area LDD1 and the second light-sensing pixels LSP2 formed in the n^{th} light-detecting area LDDn.

Although the embodiments of the disclosure have been described with reference to the accompanying drawings, those skilled in the art would understand that various modifications and alterations may be made without departing from the technical idea or essential features of the disclosure. Therefore, it should be understood that the above-mentioned embodiments are not limiting but illustrative in all aspects.

The display device may be applied to various electronic devices. The electronic device according to an embodiment may include the display device described above and may further include modules or devices having additional functions in addition to the display device.

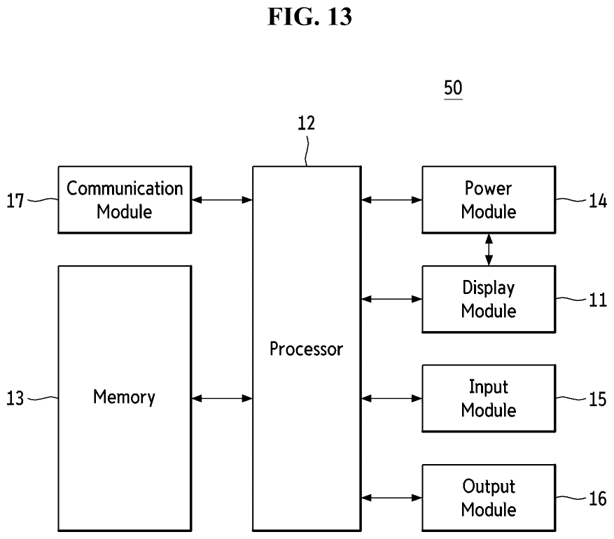

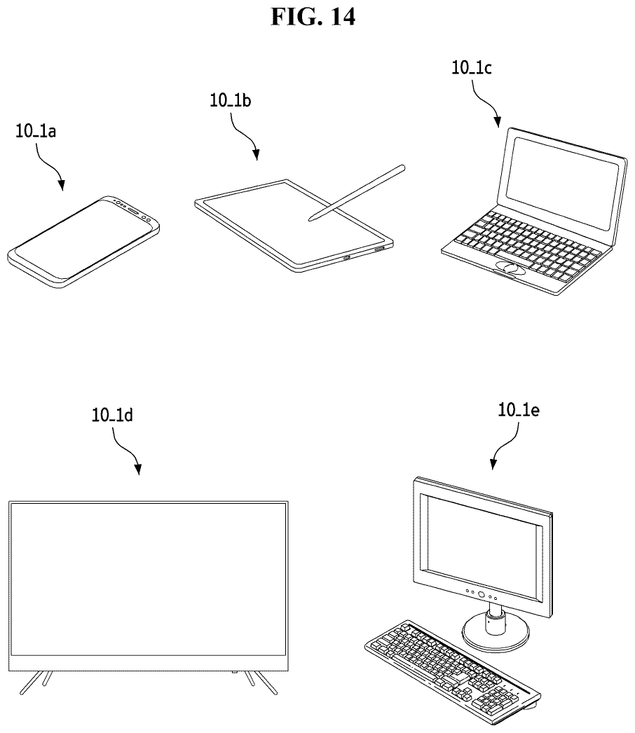

FIG. 13 is a schematic block diagram of an electronic device according to an embodiment. Referring to FIG. 13, the electronic device 50 according to an embodiment may include a display module 11, a processor 12, a memory 13, and a power module 14. The electronic device 5000 may further include an input module 15, a non-image output module 16 and/or a communication module 17.

The electronic device 50 may output various information in the form of images through the display module 11. In case that the processor 12 executes an application stored in the memory 13, image information provided by the application may be provided to the user through the display module 11. The power module 14 may include a power supply module such as a power adapter or a battery device, and a power conversion module that converts the power supplied by the power supply module to generate power required for the operation of the electronic device 50. The input module 15 may provide input information to the processor 12 and/or the display module 11. The non-image output module 16 may receive information other than images transmitted from the processor 12, such as sound, haptics, and light, and provide the information to the user. The communication module 17 may be a module that is responsible for transmitting and receiving information between the electronic device 50 and an external device, and may include a receiving unit and a transmitting unit.

At least one of the components of the electronic device 50 described above may be included in the display device described above. Some of the individual modules functionally included in one module may be included in the display device, and others may be provided separately from the display device. For example, the display device may include a display module 11, and the processor 12, memory 13, and power module 14 may be provided in the form of other devices in the electronic device 50 other than the display device.

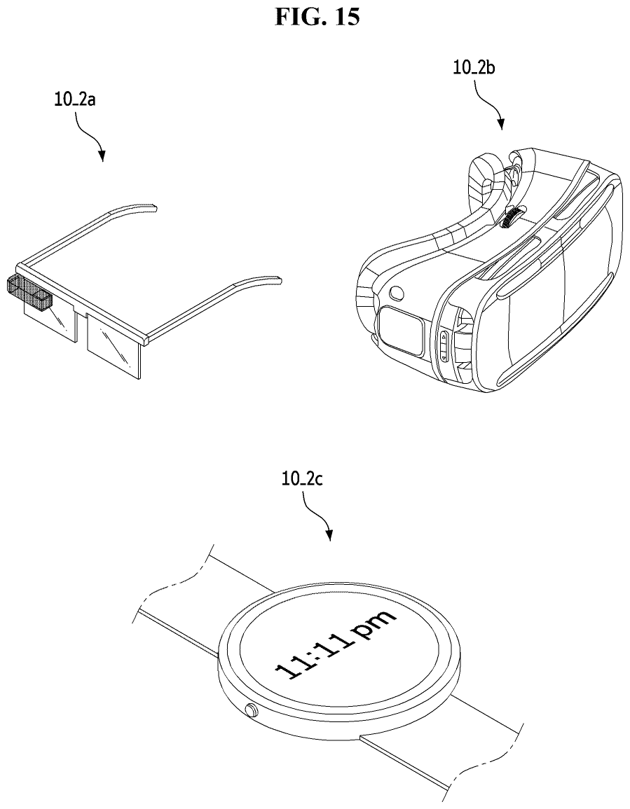

FIGS. 14, 15, and 16 are schematic diagrams of electronic devices according to various embodiments. FIGS. 14 to 16 schematically illustrate embodiments of various electronic devices to which the display device may be applied.

FIG. 14 schematically illustrates a smartphone 10_1a, a tablet PC 10_1b, a laptop 10_1c, a TV 10_1d, and a desk monitor 10_1e as embodiments of electronic devices.

In addition to the display module 11, the smartphone 10_1a may include an input module such as a touch sensor and a communication module. The smartphone 10_1a may process information received through the communication module or other input modules and display the information through the display module 11 of the display device.

The tablet PCs 10_1b, laptops 10_1c, TVs 10_1d, and desk monitors 10_1e may also include display modules 11 and input modules 15 similar to smartphones 10_1, and may additionally include communication modules in some embodiments.

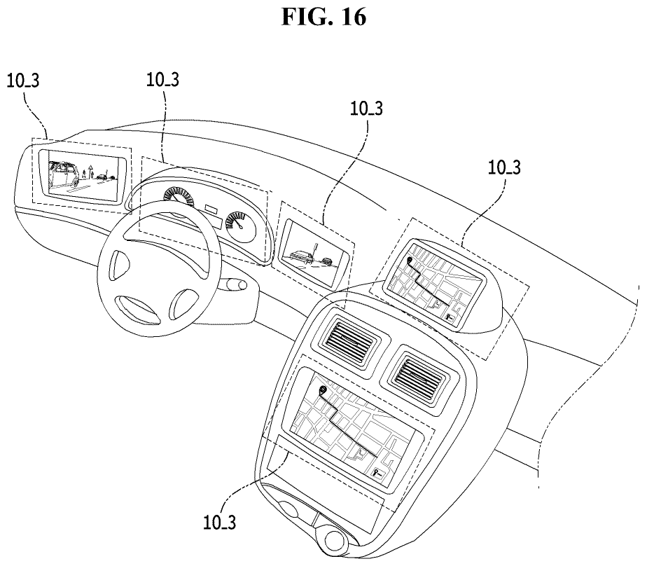

FIG. 15 shows embodiments of electronic devices including a display module being applied to a wearable electronic device. The wearable electronic device may be a smart glasses 10_2a, a head-mounted display 10_2b, a smart watch 10_2c, etc.

The smart glasses 10_2a and the head-mounted display 10_2b may include a display module that emits a display image and a reflector that reflects the emitted display image and provides the display image to the user's eyes, thereby providing a virtual reality or augmented reality image to the user.

The smart watch 10_2c may include a biometric sensor as an input device, and may provide biometric information recognized by the biometric sensor to the user through the display module. FIG. 16 schematically illustrates an embodiment that an electronic device including a display module 11 is applied to a vehicle. For example, the electronic device 10_3 may be applied to a dashboard, center fascia, etc. of a vehicle, or may be applied to a CID (Center Information Display) placed on a dashboard of a vehicle, or a room mirror display replacing a side mirror.

The above description is an example of technical features of the disclosure, and those skilled in the art to which the disclosure pertains will be able to make various modifications and variations. Therefore, the embodiments of the disclosure described above may be implemented separately or in combination with each other.

Therefore, the embodiments disclosed in the disclosure are not intended to limit the technical spirit of the disclosure, but to describe the technical spirit of the disclosure, and the scope of the technical spirit of the disclosure is not limited by these embodiments. The protection scope of the disclosure should be interpreted by the following claims, and it should be interpreted that all technical spirits within the equivalent scope are included in the scope of the disclosure.

What is claimed is:

1. A wearable display device comprising:
a housing formed in a ring or cylindrical shape;
a display panel that detects a first light-sensing signal by emitting and receiving light in a first direction and detects a second light-sensing signal by emitting and receiving light in a second direction and is disposed inside the housing in a ring or cylindrical shape; and
a main driver circuit that detects a pulse wave signal of a user, measures a biometric information of the user using the first light-sensing signal, and senses a movement of a touch by a part of the user's body using the second light-sensing signal, wherein
the first direction is an inward direction toward a center of the ring or cylindrical shape, and
the second direction is an outward direction of the housing.

2. The wearable display device of claim 1, further comprising:
a scan driver that provides gate scan signals sequentially to first and second light-emitting pixels and first and second light-sensing pixels arranged in the display panel to control light-emitting timing of the first and second light-emitting pixels and light-receiving timing of the first and second light-sensing pixels;
a circuit board electrically connected to the display panel, the main driver circuit mounted being mounted on the circuit board;
a sensing circuit disposed on the housing or the circuit board to sense a movement direction of the user and a pressure applied by the user; and
a memory that stores and sends the pulse wave signal of the user and the biometric information of the user.

3. The wearable display device of claim 1, wherein
the display panel comprises:
a biological signal detection area where at least one first light-emitting pixel that emits light in the first direction and at least one first light-sensing pixel that generates the first light-sensing signal based on an amount of light reflected from another part of the user's body in the first direction are located; and
a touch detection area comprising:
at least one light-detecting area where at least one second light-emitting pixel and at least one second light-sensing pixel are disposed; and
an image display area where a plurality of pixels for displaying images are disposed, and
the at least one second light-emitting pixel emits light in the second direction, and
the at least one second light-sensing pixel generates the second light-sensing signal based on an amount of light reflected from the part of the user's body in the second direction.

4. The wearable display device of claim 3, wherein the at least one light-detecting area is parallel to the image display area in a vertical or horizontal direction.

5. The wearable display device of claim 3, wherein
the at least one first light-sensing pixel comprises:
a photo-detecting part that outputs a light-sensing current proportional to an amount of received light incident in the first direction; and
a sense driver that provides the first light-sensing signal proportional to an amount of the light-sensing current from the photo-detecting part to the main driver circuit,
the photo-detecting part comprises a photo-detector for generating and outputting the light-sensing current proportional to the amount of the received light, and
the photo-detector provides the light-sensing current proportional to the amount of the received light in the first direction to the sense driver.

6. The wearable display device of claim 3, wherein
the at least one first light-emitting pixel comprises:
a first light-emitting unit that emits light in a green wavelength range; and
a pixel driver that applies a driving current to a first light-emitting element of
the first light-emitting unit, and
the first light-emitting unit is formed in a circular or polygonal shape in a plan view and emits light in the first direction.

7. The wearable display device of claim 6, wherein the first light-emitting unit is formed in a circular or polygonal shape in a plan view and surrounds a photo-detector formed in a circular or polygonal shape in a plan view in the at least one first light-sensing pixel.

8. The wearable display device of claim 6, wherein
the first light-emitting element of the at least one first light-emitting pixel is formed by sequentially stacking a pixel electrode, an organic material layer and a common electrode to emit light in the first direction,
the pixel electrode is formed of at least one of a reflective metal material in which aluminum and titanium are sequentially stacked, a reflective metal material in which aluminum and indium tin oxide (ITO) are sequentially stacked, and a reflective metal material in which an alloy of silver, palladium and copper, and ITO are sequentially stacked, and
the common electrode is formed of at least one of a transparent metal material comprising ITO or IZO, a semi-transparent metal material comprising at least one of magnesium and silver, and a semi-transparent metal material comprising an alloy of magnesium and silver.

9. The wearable display device of claim 3, wherein
the at least one second light-emitting pixel comprises:
a second light-emitting unit that emits light; and
a pixel driver that applies a driving current to a second light-emitting element of the second light-emitting unit, and
the second light-emitting unit is formed in a circular or polygonal shape in a plan view and emits light in the second direction.

10. The wearable display device of claim 9, wherein
the second light-emitting element of the at least one second light-emitting pixel is formed by sequentially stacking a pixel electrode, an organic material layer and a common electrode to emit light in the second direction,
the pixel electrode is formed of at least one of a transparent metal material comprising ITO or IZO, a semi-transparent metal material comprising at least one of magnesium and silver, and a semi-transparent metal material comprising an alloy of magnesium and silver, and
the common electrode is formed of at least one of a reflective metal material in which aluminum and titanium are sequentially stacked, a reflective metal material in which aluminum and indium tin oxide (ITO) are sequentially stacked, and a reflective metal material in which an alloy of silver, palladium and copper, and ITO are sequentially stacked.

11. The wearable display device of claim 3, wherein
the touch detection area comprises first and second light-detecting areas adjacent to the image display area,
the first light-detecting area is located on a side of the image display area, and the second light-detecting area is located on another side of the image display area, and
the at least one second light-emitting pixel that emits light in the second direction and the at least one second light-sensing pixel that senses light incident in the second direction are alternately arranged in the first and second light-detecting areas.

12. The wearable display device of claim 11, wherein
each of the at least one second light-emitting pixel comprises:
a second light-emitting unit that emits light; and
a pixel driver that applies a driving current to a second light-emitting element of the second light-emitting unit, and
the second light-emitting unit is formed in a circular or polygonal shape in a plan view and emits light in the second direction.

13. The wearable display device of claim 12, wherein
the second light-emitting element of the at least one second light-emitting pixel is formed by sequentially stacking a pixel electrode, an organic material layer and a common electrode to emit light in the second direction,
the pixel electrode is formed of at least one of a transparent metal material comprising ITO or IZO, a semi-transparent metal material comprising at least one of magnesium and silver, and a semi-transparent metal material comprising an alloy of magnesium and silver, and
the common electrode is formed of at least one of a reflective metal material in which aluminum and titanium are sequentially stacked, a reflective metal material in which aluminum and indium tin oxide (ITO) are sequentially stacked, and a reflective metal material in which an alloy of silver, palladium and copper, and ITO are sequentially stacked.

14. An electronic device comprising a wearable display device, wherein
the wearable display device comprises:
a housing formed in a ring or cylindrical shape;
a display panel that detects a first light-sensing signal by emitting and receiving light in a first direction and detects a second light-sensing signal by emitting and receiving light in a second direction and is disposed inside the housing in a ring or cylindrical shape; and
a main driver circuit that detects a pulse wave signal of a user, measures a biometric information of the user using the first light-sensing signal, and senses a movement of a touch by a part of the user's body using the second light-sensing signal,
the first direction is an inward direction toward a center of the ring or cylindrical shape, and
the second direction is an outward direction of the housing.

15. The electronic device of claim 14, wherein
the display panel comprises:
a biological signal detection area where at least one first light-emitting pixel that emits light in the first direction and at least one first light-sensing pixel that generates the first light-sensing signal based on an amount of light reflected from another part of the user's body in the first direction are located; and
a touch detection area comprising:
at least one light-detecting area where at least one second light-emitting pixel and at least one second light-sensing pixel are disposed; and
an image display area where a plurality of pixels for displaying images is disposed,
the at least one second light-emitting pixel emits light in the second direction, and
the at least one second light-sensing pixel generates the second light-sensing signal based on an amount of light reflected from the part of the user's body in the second direction.

16. The electronic device of claim 15, wherein
the at least one first light-emitting pixel comprises:
a first light-emitting unit that emits light in a green wavelength range; and
a pixel driver that applies a driving current to a first light-emitting element of the first light-emitting unit, and
the first light-emitting unit is formed in a circular or polygonal shape in a plan view and emits light in the first direction.

17. The electronic device of claim 16, wherein
the first light-emitting element of the at least one first light-emitting pixel is formed by sequentially stacking a pixel electrode, an organic material layer and a common electrode to emit light in the first direction,
the pixel electrode is formed of at least one of a reflective metal material in which aluminum and titanium are sequentially stacked, a reflective metal material in which aluminum and indium tin oxide (ITO) are sequentially stacked, and a reflective metal material in which an alloy of silver, palladium and copper, and ITO are sequentially stacked, and
the common electrode is formed of at least one of a transparent metal material comprising ITO or IZO, a semi-transparent metal material comprising at least one of magnesium and silver, and a semi-transparent metal material comprising an alloy of magnesium and silver.

18. The electronic device of claim 15, wherein
the at least one second light-emitting pixel comprises:
a second light-emitting unit that emits light; and
a pixel driver that applies a driving current to a second light-emitting element of the second light-emitting unit, and
the second light-emitting unit is formed in a circular or polygonal shape in a plan view and emits light in the second direction.

19. The electronic device of claim 18, wherein
the second light-emitting element of the at least one second light-emitting pixel is formed by sequentially stacking a pixel electrode, an organic material layer and a common electrode to emit light in the second direction,
the pixel electrode is formed of at least one of a transparent metal material comprising ITO or IZO, a semi-transparent metal material comprising at least one of magnesium and silver, and a semi-transparent metal material comprising an alloy of magnesium and silver, and
the common electrode is formed of at least one of a reflective metal material in which aluminum and titanium are sequentially stacked, a reflective metal material in which aluminum and indium tin oxide (ITO) are sequentially stacked, and a reflective metal material in which an alloy of silver, palladium and copper, and ITO are sequentially stacked.

20. The electronic device of claim 15, wherein
the touch detection area comprises first and second light-detecting areas adjacent to the image display area,
the first light-detecting area is located on a side of the image display area, and the second light-detecting area is located on another side of the image display area, and
the at least one second light-emitting pixel that emits light in the second direction and the at least one second light-sensing pixel that senses light incident in the second direction are alternately arranged in the first and second light-detecting areas.
